(19) United States
(12) Patent Application Publication
(10) Pub. No.: US 2021/0046205 A1
(43) Pub. Date: Feb. 18, 2021

(54) AUTONOMOUS ROBOT TO REMOVE PATHOGENS FROM A TARGET AREA

(71) Applicant: Patten Seed Company, Lakeland, GA (US)

(72) Inventors: BENJAMIN COPELAND, JR., FORT VALLEY, GA (US); NICK SAGNELLA, CHARLOTTE, NC (US)

(73) Assignee: Patten Seed Company, Lakeland, GA (US)

(21) Appl. No.: 16/542,660

(22) Filed: Aug. 16, 2019

(51) Int. Cl. A61L 2/24; H02J 7/02; A61L 2/10; G05D 1/00; G05D 1/02

(52) U.S. Cl. CPC A61L 2/24; H02J 7/022; A61L 2/10; G05D 1/0094; G05D 1/0278; G05D 2201/0201; G05D 1/0088; G05D 1/0265; A61L 2202/14; A61L 2202/11; A61L 2202/16; G05D 1/0225

(57) ABSTRACT

Disclosed is an autonomous robot to remove pathogens from a target area such as a grass field, turf field, or yard. Embodiments include an ultraviolet light subsystem that generates ultraviolet radiation operable to remove the pathogens from the target area as the autobot advances along the coverage path.

### TECHNICAL FIELD

The present invention relates to an autonomous robot, in particular to an autonomous robot to remove pathogens from a target area.

### BACKGROUND

Ultraviolet (“UV”) light may be used to remove or eliminate microorganisms, pathogens, algae, and fungi in various environmental applications. Moreover, UV light treatments are incorporated in air purification, water purification, aquarium and pond maintenance, laboratory hygiene and food and beverage protection systems known in the art.

Generally, for systems known in the prior art, UV treatment is performed inside a specialized UV exposure chamber to eliminate microorganisms in air and water. Embodiments of the solution proposed in this specification, however, recognize that the usage of UV treatment for an outdoor environment, such as a grass field, can be an efficient and safer alternative to other treatment methods, particularly chemical-based treatment methods, because UV treatment does not produce any residual chemical or radiation in the air or water and is harmless to untargeted organisms and plants.

U.S. Pat. No. 8,911,664 B1 to Cavanaugh discloses a method for delivering UV light to plants, crops, and ornamentals for the purposes of killing pathogens. Notably, however, the Cavanaugh system is complex, requires manual propulsion and is impractical, if not altogether unsuitable, for use during night hours. Notably, pathogenic fungal mycelium growth in grass is greatest at night. The sun provides the UV light during the day to check fungal growth and, therefore, manual methods delivering UV light treatment to a grass area are ineffective because they are necessarily implemented in daylight hours.

Therefore, there is a need for an efficient, effective, and intelligent autonomous robot (autobot) to remove pathogens present in a target area, such as a grass field, in low light or no light conditions. Moreover, there is a need in the art for a system that delivers UV light treatment to a target area, such as a grass field, with minimal labor costs and environmental impact.

Thus, in view of the above, there is a long-felt need in the industry to address the aforementioned deficiencies and inadequacies. Further limitations and disadvantages of conventional and traditional approaches will become apparent to one of skill in the art through comparison of the prior art approaches with aspects of the solution in the present disclosure, as set forth in the remainder of the present application and with reference to the drawings.

### SUMMARY

The present solution cures and solves technical problems existing in the prior art. In response to these problems, the present solution provides an autonomous robot to remove pathogens from a target area. An first exemplary embodiment of the solution includes an autonomous robot (autobot) configured to remove pathogens from a target area. The autobot comprises a body portion to support a plurality of components of the autobot comprised of an AC/DC converter to transform alternating current (AC) input received from an electrical power supply into direct current (DC) output, a battery to store the direct current (DC) output (wherein the electrical power supply is connected to a charge station to recharge the battery when the autobot is docked at the charge station), a processor mounted on the body portion to store a plurality of position markers and a plurality of executable instructions pertaining to a coverage path of the target area (wherein the position markers are a plurality of underground reference points positioned along the coverage path), a sensing system operative to sense the presence of the position markers and transmit data signals associated with the position markers to the processor (wherein the processor operates in response to a received data signal from the sensing system to compare the received data signal with the stored position markers to determine an actual position of the autobot within the target area), a controller connected to the processor for controlling the movement of the autobot along the coverage path (wherein the processor transmits a correction signal to the controller in response to identifying a deviation of the actual position from the coverage path and wherein the controller adjusts movement of the autobot on receiving the correction signal), an ultraviolet light subsystem connected to the processor to generate ultraviolet, type C (UVC) radiation with a predefined power output operable to remove the pathogens from the target area as the autobot advances along the coverage path (wherein the processor stores one or more of UVC radiation data and removed pathogens data), and a transmitter connected to the processor and operable to wirelessly transmit the UVC radiation data and/or removed pathogens data to a remote computing device.

The controller in the first exemplary embodiment of an autobot initiates a return signal to the autobot on detecting a battery level signal that is below a predefined battery level and returns the autobot to the charge station. The first exemplary embodiment of an autobot may further comprise a dusk-dawn sensor mounted on the body portion to determine an optimal duration to remove the pathogens from a surface of the target area. The first exemplary embodiment of an autobot may further comprise a Global Positioning System (GPS) connected to the sensing system to establish a geo-fence within which generation of ultraviolet, type C (UVC) radiation occurs. The first exemplary embodiment of an autobot may further comprise an intensity controller connected to adjust a power output of the ultraviolet light subsystem. The first exemplary embodiment of an autobot may further comprise a battery operable to transmit a battery level signal to the controller.

A second exemplary embodiment of the solution includes an autonomous robot (autobot) configured to remove pathogens from a target area defined by a geo-fence. The autobot comprises a body portion to support a plurality of components of the autobot comprised of an AC/DC converter to transform alternating current (AC) input received from an electrical power supply into direct current (DC) output, a battery to store the direct current (DC) output (wherein the electrical power supply is connected to a charge station to recharge the battery when the autobot is docked at the charge station), a Global Positioning System (GPS) module operable to generate a plurality of GPS coordinates representing a geographical location of the autobot within the target area defined by the geo-fence, a controller connected to the GPS module and comprising a processor and a memory (wherein the controller is configured to compare the geographical location of the autobot with the GPS coordinates associated with the geo-fence and, based on the comparison, control the movement of the autobot within the target area according to an algorithm for defining a coverage path of the target area), and an ultraviolet light subsystem configured to generate ultraviolet, type C (UVC) radiation with a predefined power output operable to remove the pathogens from the target area as the autobot advances along the coverage path.

The processor in the second exemplary embodiment of an autobot may be further configured to store in the memory one or more of UVC radiation data and removed pathogens data. The second exemplary embodiment of an autobot may further comprise a transmitter connected to the processor and operable to wirelessly transmit the UVC radiation data and/or removed pathogens data to a remote computing device. The second exemplary embodiment of an autobot may further comprise a timer module in communication with the controller (wherein the timer module is configured to generate timer signals defining an optimal duration for pathogen removal from the target area and the controller is further configured to, based on the timer signals, cause the autobot to advance along the coverage path or dock to the charge station). The second exemplary embodiment of an autobot may further comprise a dusk-dawn sensor in communication with the timer module. The controller in the second exemplary embodiment of an autobot may be further configured to monitor a battery charge level and, upon determining that the battery charge level is below a predefined threshold, cause the autobot to dock at the charge station.

A third exemplary embodiment of the solution includes an autonomous robot (autobot) configured to remove pathogens from a target area defined by a geo-fence. The autobot comprises a body portion to support a plurality of components of the autobot comprised of a body portion to support a plurality of components comprised of an AC/DC converter to transform alternating current (AC) input received from an electrical power supply into direct current (DC) output, a battery to store the direct current (DC) output (wherein the electrical power supply is connected to a charge station to recharge the battery when the autobot is docked at the charge station), a sensing system operative to sense the presence of a boundary wire defining the perimeter of the target area and a charge station guide wire dissecting the target area, a controller connected to the sensing system and comprising a processor and a memory and configured to control the movement of the autobot within the target area according to an algorithm for defining a coverage path of the target area (wherein the controller is configured to receive signals from the sensing system and, based on the signals, adjust a movement direction of the autobot according to the algorithm), and an ultraviolet light subsystem configured to generate ultraviolet, type C (UVC) radiation with a predefined power output operable to remove the pathogens from the target area as the autobot advances along the coverage path.

The processor in the third exemplary embodiment of an autobot may be further configured to store in the memory component one or more of UVC radiation data and removed pathogens data. The third exemplary embodiment of an autobot may further comprise a transmitter connected to the processor and operable to wirelessly transmit the UVC radiation data and/or removed pathogens data to a remote computing device. The third exemplary embodiment of an autobot may further comprise a timer module in communication with the controller (wherein the timer module is configured to generate timer signals defining an optimal duration for pathogen removal from the target area and the controller is further configured to, based on the timer signals, cause the autobot to advance along the coverage path or dock to the charge station). The third exemplary embodiment of an autobot may further comprise a dusk-dawn sensor in communication with the timer module. The controller in the third exemplary embodiment of an autobot may be further configured to monitor a battery charge level and, upon determining that the battery charge level is below a predefined threshold, cause the autobot to dock at the charge station by directing movement of the autobot along the guide wire.

The above-described and additional features may be considered, and will become apparent in conjunction with the drawings, in particular, and the detailed description that follows.

### BRIEF DESCRIPTION OF THE DRAWINGS

In the figures, similar components and/or features may have the same reference label. Further, various components of the same type may be distinguished by following the reference label with a second label that distinguishes among the similar components. If only the first reference label is used in the specification, the description applies to any one of the similar components having the same first reference label irrespective of the second reference label.

FIG. 1 illustrates a functional block diagram of an autonomous robot (“autobot”) configured to remove pathogens from a target area, in accordance with one embodiment of the solution;

FIG. 2 illustrates a first functional block diagram of an autonomous robot (“autobot”) configured to remove pathogens from a target area defined by a geo-fence, in accordance with one embodiment of the solution;

FIG. 3 illustrates a second functional block diagram of an autonomous robot (“autobot”) configured to remove pathogens from a target area defined by a geo-fence, in accordance with one embodiment of the solution;

FIG. 4 illustrates an operational view of an autonomous robot (“autobot”) configured to remove pathogens from a target area defined by a geo-fence, in accordance with one embodiment of the solution;

FIG. 5 illustrates a perspective view of an exemplary autonomous robot (“autobot”) according to the solution;

FIG. 6 illustrates an assembled view of an autonomous robot (“autobot”) according to the solution comprising an exemplary ultraviolet light subsystem removably mounted to the body of the autobot;

FIG. 7 illustrates an assembled view of an autonomous robot (“autobot”) according to the solution comprising an exemplary ultraviolet light subsystem removably mounted to the underside of the autobot body; and

FIG. 8 illustrates an assembled view of an AC/DC converter and a battery comprised within an exemplary embodiment of an autonomous robot (“autobot”) according to the solution.

### DETAILED DESCRIPTION

The present solution is best understood with reference to the detailed figures and description set forth herein. Various exemplary embodiments are discussed with reference to the figures. Those skilled in the art, however, will readily appreciate that the detailed descriptions provided herein with respect to the figures are offered merely for explanatory purposes and do not necessarily encompass the entirety of the novel solution, as the methods and systems may extend beyond the described embodiments. For instance, the teachings presented herein and the needs of a particular application may yield multiple alternative and suitable approaches to implement the functionality of any detail described herein. Therefore, any approach may extend beyond certain exemplary implementation choices in the following embodiments.

Embodiments of the solution include an autonomous robot (“autobot”) with an ultraviolet light emitting subsystem. As will be explained in more detail below, the autobot is self-propelled and configured to systematically traverse a target area, such as a grass field or lawn, while the ultraviolet light emitting subsystem exposes the target area to UV light. Advantageously, in this way embodiments of the solution may kill or remove pathogens from the target area via exposure to the UV light. It is a further advantage of embodiments of the solution that the autobot may traverse the target area without manual propulsion and, therefore, may do so efficiently without the benefit of an ambient light source (such as at night). Moreover, because embodiments of the solution may traverse the target area in the dark, it is envisioned that the efficacy for pathogen removal via UV light may be improved over prior art solutions.

Embodiments of the present solution include various steps, which will be described below. Although the present solution is described with the purpose of removing pathogens from a target area such as a grass field, it should be appreciated that the same has been done merely to illustrate the solution in an exemplary manner and to highlight any other purpose or function for which explained structures or configurations could be used and is covered within the scope of the present invention.

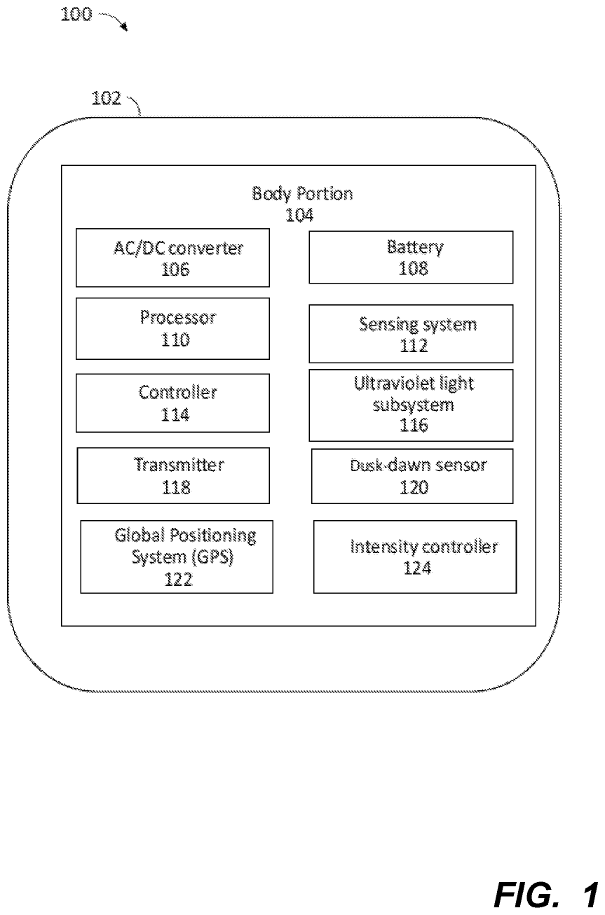

FIG. 1 illustrates a functional block diagram 100 of an autonomous robot (“autobot”) 102 configured to remove pathogens from a target area, in accordance with one embodiment of the solution. The autobot 102 includes a body portion 104, including a frame, to support various components of the autobot. In an exemplary embodiment, the autobot is in the form of an autonomous lawn mower, although it will be understood that not all autobots 102 according to the solution include mowing functionality or, for that matter, any functionality applicable to the target area beyond application of UV light via subsystem 116. The components mounted within or on body portion 104 may comprise one or more of an AC/DC converter 106, a rechargeable battery 108, a processor 110, a sensing system 112, a controller 114, an ultraviolet light subsystem 116, a transmitter 118, a dusk-dawn sensor 120, a Global Positioning System (GPS) 120, and an intensity controller 124. Additionally, although not depicted in the FIG. 1 illustration, it will be understood that embodiments of an autobot 102 according to the solution may include a powertrain having one or more of an electric motor, wheels (or some other means for movement across the target area), axle(s), gearing, drives, etc.

The AC/DC converter 106 transforms alternating current (AC) input received from an electrical power supply (not shown in the FIG. 1 illustration) into direct current (DC) output. The battery 108 stores the direct current (DC) output, as would be understood by one of ordinary skill in the art. The electrical power supply is connected to a charge station to recharge the battery 108 when the autobot 102 is docked at the charge station. Energy stored in the battery 108 may be used to drive an electric motor and power the UV subsystem 116 as well as other components within the autobot 102. In an embodiment, the battery 108 transmits a battery level signal to the controller 114. In an exemplary embodiment, the battery 108 may include a battery such as lithium ion battery or batteries having small current ratings with a long discharging cycle, such as an absorbent glass mat (AGM) battery, a sealed lead acid (SLA) battery, a flooded lead-acid battery, or a gel cell. Even so, it will be understood that embodiments of the solution are not limited to any particular type of battery, as one of ordinary skill in the art will be able to select a battery type and size in view of factors including, but not necessarily limited to, the size/weight of the autobot 102, the current draw requirements of the subsystem 116, the size of the target area to be covered between charging events, etc.

In an exemplary embodiment, the processor 110 may store data associated with various position markers in the target area and executable instructions pertaining to a coverage path of the target area. The position markers may be a plurality of underground reference points positioned along the desired coverage path. Examples of the processor 110 include, but are not limited to, special-purpose processing devices such as an application specific integrated circuit (ASIC), a digital signal processor (DSP), etc. Additionally, reference to the processor 110 may represent general-purpose processing devices such as a microprocessor, a central processing unit, etc. Further, reference to the processor 110 will be understood to include a memory component for storage of executable instructions, reference data, etc., as would be understood by one of ordinary skill in the art of electronics.

The sensing system 112 senses the presence of a position marker and transmits to the processor 110 a data signal in accordance therewith upon sensing the position marker. The processor 110 operates in response to the data signal received from the sensing system 112 to compare the data signal with the stored position markers in order to determine an actual position within the target area.

In view of the position marker sensed by the sensing system 112 and determined by the processor 110 based on the stored data associated with the position markers, the controller 114 controls and directs the movement of the autobot 102 along the desired coverage path of the target area. In this way, an embodiment of the solution that leverages position markers may systematically traverse the target area according to a predefined coverage path.

In other embodiments of the solution that leverage position markers, a visual positioning system that comprises a camera subsystem may be included in the sensing system 112. The visual positioning system, working with the processor 110, may be configured to determine the location of the camera-enabled autobot by decoding location coordinates from visual markers strategically placed around the target area and recognized by the camera subsystem. In such an application, markers are placed at specific locations throughout the target area, each marker encoding that location's positional coordinates: latitude, longitude and height off the ground. Measuring the visual angle from the autobot to the marker enables the processor to estimate the autobot's own location coordinates in reference to the marker. In this way, the autobot may manipulate the controller 114 to navigate the autobot around and within the target area.

In other embodiments of the solution, the sensing system 112 and/or GPS module 122 may comprise an inertial measurement unit (“IMU”) that includes, inter alia, accelerometer(s) and gyroscope(s). An IMU may be integrated into a GPS based navigation system to achieve a dead reckoning capability and the ability to gather data indicative of the autobot's current speed, turn rate, heading, inclination and acceleration. The data generated by the IMU is communicated to the processor 110 which may use the data to calculate attitude, velocity and position of the UV autobot 100. An exemplary implementation of an IMU understood in the art may take the form of a Strap Down Inertial System that integrates angular rate from the gyroscope to calculate angular position. The angular position may be fused with the gravity vector measured by the accelerometers in a Kalman filter to estimate attitude. The attitude estimate may then be used to transform acceleration measurements into an inertial reference frame where they are integrated once to get linear velocity, and twice to get linear position.

For example, if an IMU comprised within a UV autobot moving along a certain direction vector were to measure the autobot's acceleration as 0.5 m/s² for 1 second, then after that 1 second the processor may calculate that the autobot must be traveling at 0.5 m/s and must be 0.25 m from its initial position (assuming v₀=0 and known starting position coordinates x₀, y₀, z₀). If combined with a digital map archive of the target area stored in the memory 112, the autobot may remotely transmit the location data to a central system to indicate where the autobot is located geographically within the target area in a certain moment, as with a GPS navigation system alone—but without the need to communicate with or receive communication from any outside components, such as satellites or land radio transponders, though external sources such as GPS may still be used by the autobot processor 110 in order to correct drift errors (as would be understood by one of ordinary skill in the art of positional tracking systems). This method of navigation is called dead reckoning.

Certain other embodiments of the solution may leverage Timing & Inertial Measurement Unit (“TIMU”) ICs that are configured to conduct absolute position tracking on a single chip without GPS-aided navigation. As would be understood by one of ordinary skill in the art, a TIMU IC chip integrates a master timing clock into an IMU chip. An exemplary TIMU chip may include an integrated 3-axis gyroscope, a 3-axis accelerometer, and a 3-axis magnetometer. Together with the master timing clock, an exemplary TIMU chip may simultaneously measure the tracked movement of the associated autobot and combine the measurement with timing from the synchronized clock. Fusing the measurements, absolute position tracking of the autobot may be realized without the need to leverage external transmitters or transceivers (such as GPS).

In certain embodiments, the controller 114 may initiate a return signal to the autobot 102 on detecting that a battery level signal is below a predefined battery level and, in doing so, causes the autobot 102 to return to the charge station. The processor 110 may transmit a correction signal to the controller 114 in response to identifying a deviation of the actual position of the autobot 102 in the target area from the desired coverage path or return path. The controller 114 may adjust the directional movement of the autobot 102 on receiving the correction signal. As the autobot 102 traverses the target area along the coverage path, the ultraviolet light subsystem 116 may generate ultraviolet, type C (UVC) radiation with a predefined power output operable to remove pathogens. In certain embodiments, the processor 110 may store one or more of UVC radiation data and removed pathogens data.

The transmitter 118 is communicatively connected to the processor 110 and may be operable to wirelessly transmit the UVC radiation data and/or removed pathogens data to a remote computing device. The wireless communication between the transmitter 118 and a remote computing device, established by using wireless technologies, may include, but is not limited to, cellular protocols, SMS, MMS, short-wave radio protocols (e.g., Bluetooth), Wi-Fi, and Infrared. Examples of the remote computing device may include, but are not limited to, personal computers, laptops, personal digital assistants (PDAs), mobile devices, tablets, or any other computing device.

A dusk-dawn sensor 120 mounted on the body portion 104 may be leveraged to determine an optimal duration or time window to remove the pathogens from the target area. In this way, an autobot 102 according to the solution may depart from its docking/charging station for application of UV light to the target area when the sensor 120 indicates to the processor 110 that ambient light is below (or above) a predefined level.

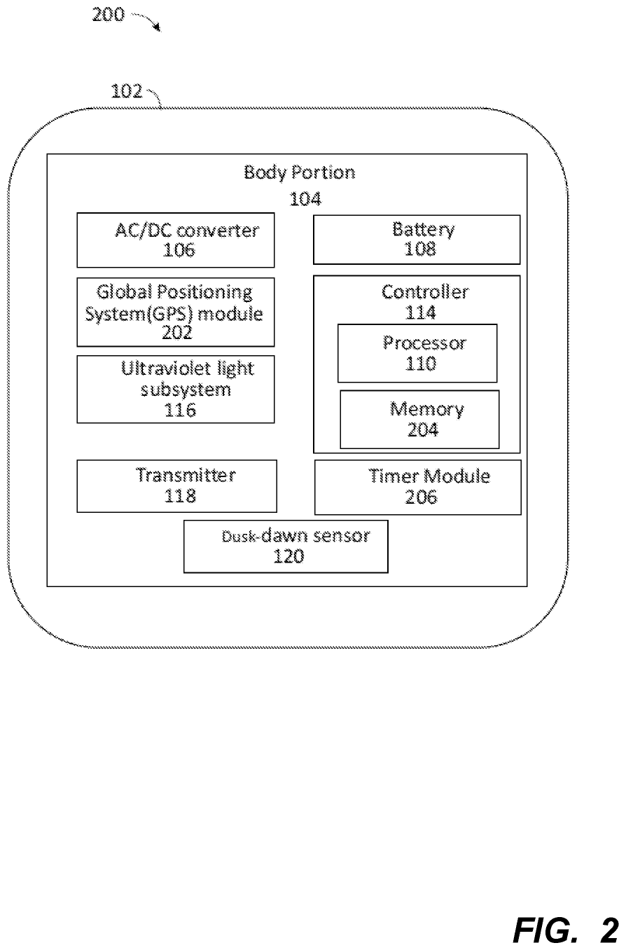

A Global Positioning System (GPS) 122 may be connected to the sensing system 112 and leveraged to establish a geo-fence within which generation of ultraviolet, type C (UVC) radiation occurs. In this way, the target area may be predefined using a geo-fence of GPS coordinates such that the GPS 122, working with the sensing system 112 and/or processor 110 and controller 114, recognizes boundaries of the target area. Similarly, in embodiments of the soluton that leverage a GPS 122, the aforementioned coverage path within the target area may be predefined by a series of GPS coordinates. In such an embodiment, the markers may be associated with specific GPS coordinates in lieu of markers physically sensed by the sensing system 112. It is further envisioned that embodiments of the solution may not use a predefined coverage path and, instead, may traverse the target area according to an algorithm that causes the controller 114 to randomly redirect the direction of movement within the geo-fence (whether the geo-fence is defined by GPS coordinates or a physical barrier or physical position indicators capable of being sensed by the sensing system 112), thereby statistically covering the entire target area over time. An embodiment of the solution that leverages GPS coordinates for defining a geo-fence and/or a coverage path within the target area will be described in more detail relative to the FIG. 2 illustration.

The FIG. 1 illustration further includes an intensity controller 124. It is envisioned that certain embodiments of the solution may include an intensity controller 124 that is operable to adjust a power output of the ultraviolet light subsystem 116. Depending on embodiment, the intensity controller 124 may adjust the power output (i.e., the intensity of the UV light output) of the ultraviolet light subsystem 116 in view of one or more triggers such as, but not limited to, ambient light levels, timing, location within the target area, and target area type (e.g., grass type in the target area, grass height, etc.). Moreover, it is envisioned that the intensity controller 124 may adjust the power output of the subsystem 116 according to user defined parameters.

FIG. 2 illustrates a first functional block diagram 200 of an autonomous robot (“autobot”) 102 configured to remove pathogens from a target area defined by a geo-fence, in accordance with one embodiment of the solution. The autobot 102 includes a body portion 104 to support various components of the autobot, as previously described relative to the FIG. 1 illustration. The various components shown in the FIG. 2 illustration include an AC/DC converter 106, a rechargeable battery 108, a Global Positioning System (GPS) module 202, a controller 114, an ultraviolet light subsystem 116, a transmitter 118, a timer module 206, and a dusk-dawn sensor 120. The AC/DC converter 106 transforms alternating current (AC) input received from an electrical power supply into direct current (DC) output that is stored in battery 108, as would be understood by one of ordinary skill in the art. The electrical power supply is connected to a charge station to recharge the battery 108 when the autobot is docked at the charge station, as previously described. Energy stored in the battery 108 may be used to drive an electric motor and power the UV subsystem 116 as well as other components within the autobot 102. The Global Positioning System (GPS) module 202 may be operable to generate a plurality of GPS coordinates representing a geographical location of the autobot 102 within the target area defined by a geo-fence. The controller 114 may be communicatively connected to the GPS module 202.

The controller 114 includes a processor 110 and a memory 204. The controller 114 may be configured to compare the geographical location of the autobot 102 with the GPS coordinates associated with the geo-fence and, based on the comparison, control the movement of the autobot 102 within the target area according to an algorithm for defining a coverage path of the target area. It is envisioned that the coverage path may be predefined (e.g., a coverage path defined by a series of adjacent rows) or randomly determined (e.g., a random change of direction such that the target area is eventually traversed in its entirety while certain areas within the target area are traversed a plurality of times as the movement of the autobot causes its path to crisscross). Algorithms and predefined patterns for determining a coverage path will occur to those of skill in the art. In an embodiment, the processor 110 is further configured to store in the memory 204 one or more of UVC radiation data and removed pathogens data.

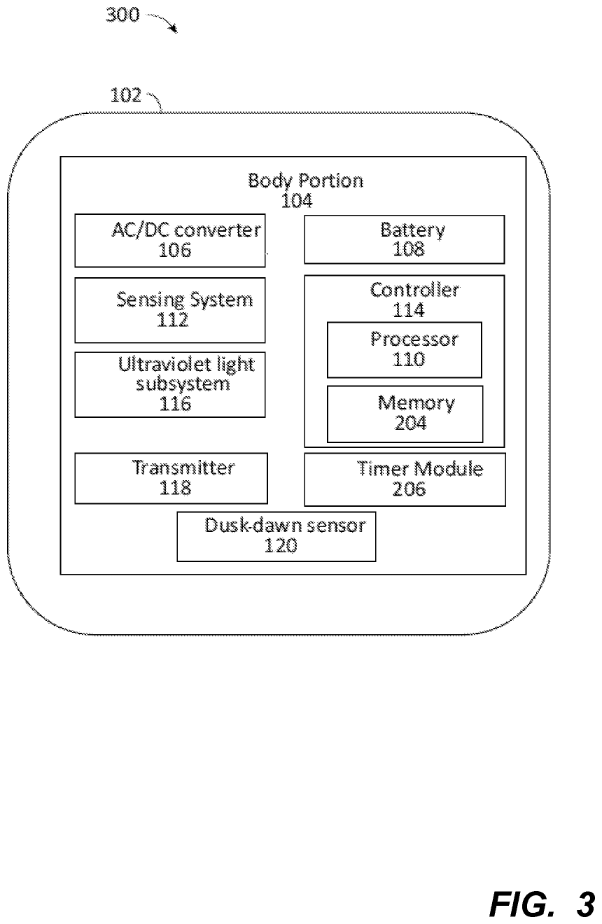

FIG. 3 illustrates a second functional block diagram 300 of an autonomous robot (“autobot”) configured to remove pathogens from a target area defined by a geo-fence, in accordance with one embodiment of the solution. The autobot 102 includes a body portion 104 to support various components of the autobot, as previously described relative to the FIG. 1 illustration. The various components include an AC/DC converter 106, a rechargeable battery 108, a sensing system 112, a controller 114, an ultraviolet light subsystem 116, a transmitter 118, a timer module 206, and a dusk-dawn sensor 120.

The AC/DC converter 106 transforms alternating current (AC) input received from an electrical power supply into direct current (DC) output. The AC/DC converter 106 transforms alternating current (AC) input received from an electrical power supply into direct current (DC) output that is stored in battery 108, as would be understood by one of ordinary skill in the art. The electrical power supply is connected to a charge station to recharge the battery 108 when the autobot is docked at the charge station, as previously described. Energy stored in the battery 108 may be used to drive an electric motor and power the UV subsystem 116 as well as other components within the autobot 102.

The sensing system 112 may be operative to sense the presence of a boundary wire defining the perimeter of the target area and a charge station guide wire dissecting the target area. The controller 114 is connected to the sensing system 112. The controller 114 includes a processor 110 and a memory 204 and is configured to control the movement of the autobot 102 within the target area according to an algorithm for defining a coverage path of the target area, as previously described. In an embodiment, the processor 110 may be further configured to store in the memory 204 one or more of UVC radiation data and removed pathogens data.

The controller 114 may be configured to receive signals from the sensing system 112 and, based on the signals, adjust a movement direction of the autobot 102 according to the algorithm. The ultraviolet light subsystem 116 may be configured to generate ultraviolet, type C (UVC) radiation with a predefined power output useful for removing pathogens from the target area as the autobot 102 advances along the coverage path. The transmitter 118 may be connected to the processor 110 and operable to wirelessly transmit the UVC radiation data and/or removed pathogens data to a remote computing device.

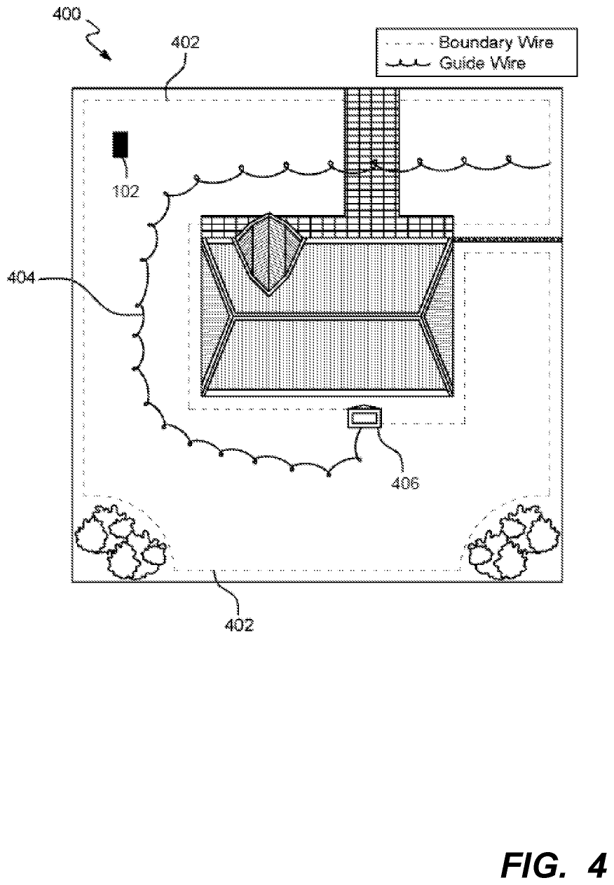

The timer module 206 may be in communication with the controller 114 and configured to generate timer signals defining an optimal duration for pathogen removal from the target area. The controller 114 may be further configured to, based on the timer signals, cause the autobot to advance along the coverage path or dock to the charge station. As will be described in more detail relative to the FIG. 4 illustration, the autobot 102 may find its way back to the docking station be recognizing the presence of the aforementioned guide wire. In an embodiment, the controller 114 may be configured to monitor a battery charge level and, upon determining that the battery charge level is below a predefined threshold, cause the autobot to dock at the charge station by directing movement of the autobot along the guide wire.

FIG. 4 illustrates an operational view 400 of an autonomous robot (“autobot”) configured to remove pathogens from a target area defined by a geo-fence 402, in accordance with one embodiment of the solution. FIG. 4 shows that the boundary wire 402 (which defines the geo-fence in the particular application illustrated by FIG. 4—other applications may leverage GPS coordinates, Wi-Fi broadcast ranges, or other means for defining the geo-fence) is buried around the target area and a guide wire 404 is buried such that it strategically dissects the target area. The sensing system 112 placed on the autobot 102 detects the boundary wire 402 and works with the controller 114 to change the direction of the autobot 102 to stay within the geo-fence 402. In an embodiment, the path algorithm may be predefined or random. When the battery 108 needs a charge, upon detecting the guide wire 404 the autobot 102 will follow the guide wire 404 back to the charging station 406.

Thus an exemplary embodiment of the present solution provides an autobot 102 that utilizes a sensing system 112 that keys off of the boundary wire 402 that defines the perimeter of a yard or grass field. The autobot 102 stays within the perimeter and leverages the algorithm to traverse the yard while the UV subsystem emits pathogen-killing light. The autobot 102 automatically returns to a charging station 406 when it needs a charge. In an embodiment, the autobot 102 leverages GPS to stay within the geofence that defines the yard or grass field instead of relying on the recognition of a boundary wire 402.

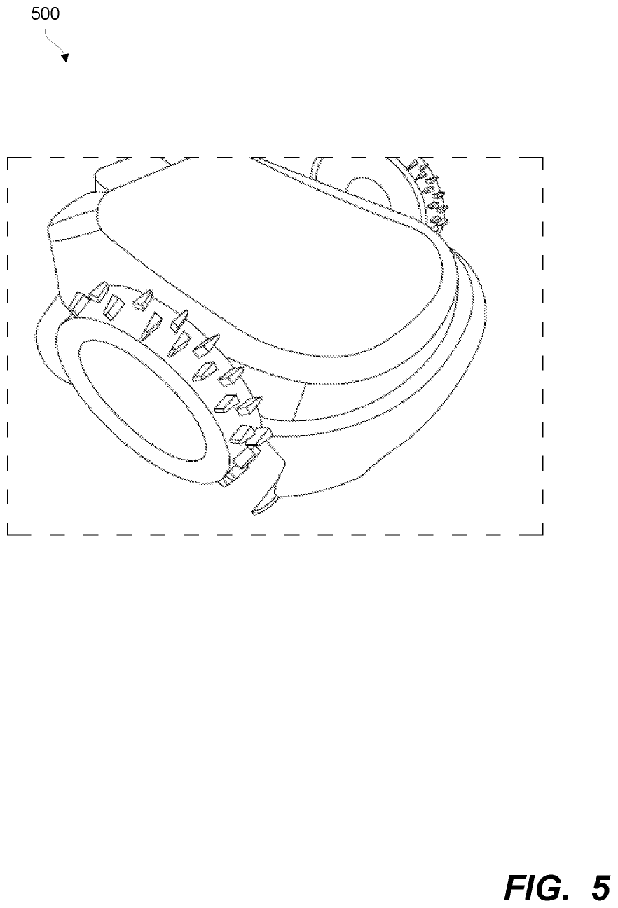

FIG. 5 illustrates a perspective view of an exemplary autonomous robot (“autobot”) 500 according to the solution. As can be understood from the FIG. 5 illustration, the autobot 500 includes a pair of wheels. Each wheel may be driven independently from the other, thereby allowing the controller to change direction of movement for the autobot 500 by varying the rotational speed of one wheel relative to the other. The UV light-emitting subsystem may be incorporated within the exemplary autobot 500 beneath the body such that it is not exposed outside of the body perimeter. In this way, application of the UV light to the target area may be improved in high ambient light conditions.

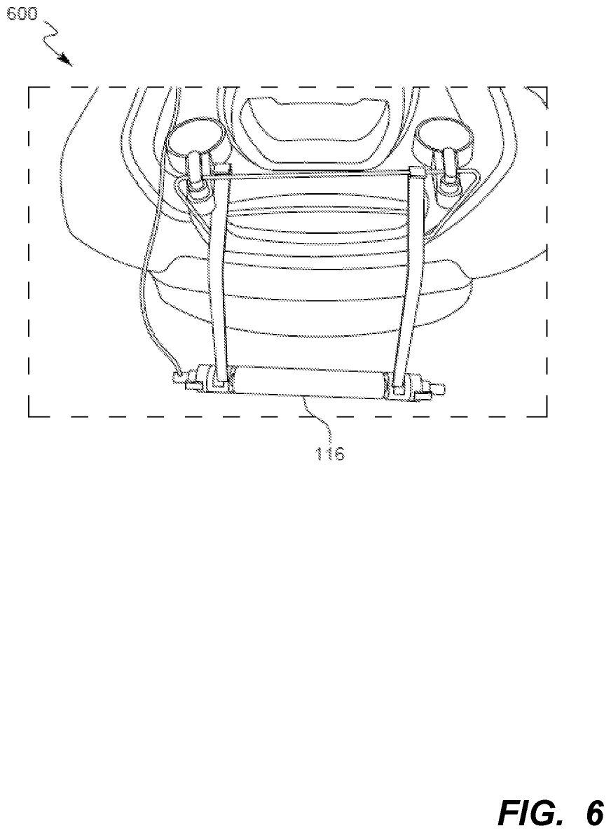

FIG. 6 illustrates an assembled view of an autonomous robot (“autobot”) 600 according to the solution comprising an exemplary ultraviolet light subsystem 116 that is removably mounted external to the autobot body. The exemplary ultraviolet light subsystem 116 shown in the FIG. 6 illustration includes a single ultraviolet bulb removably mounted such that it is suspended out ahead of the autobot. The UV light subsystem 116 is powered by the battery 108, as previously described. The ultraviolet light subsystem 116 is configured to generate ultraviolet, type C (UVC) radiation with a predefined power output (or a variable power output, in some embodiments), as previously described, and is operable to remove the pathogens from the target area as the autobot advances along a coverage path.

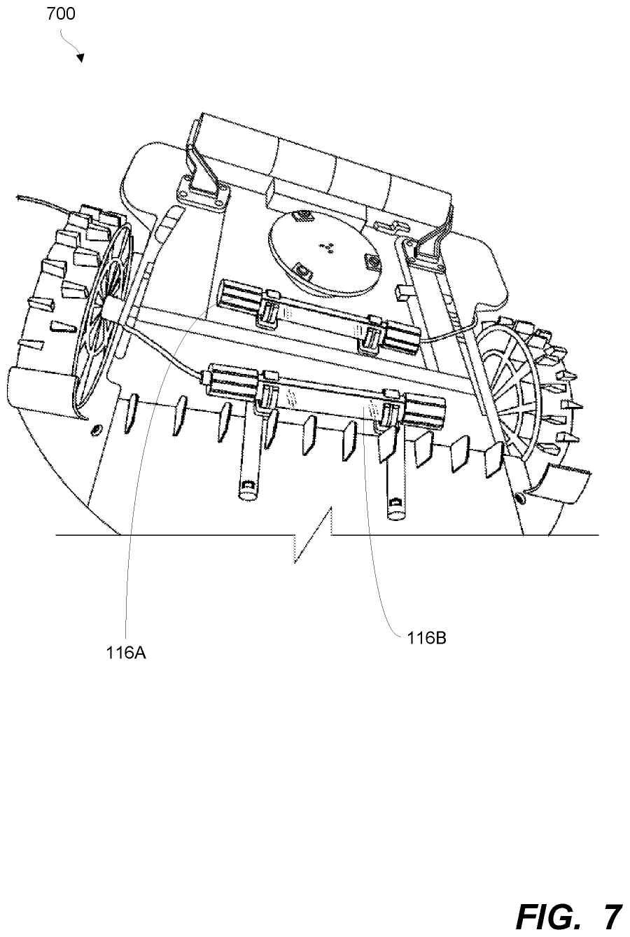

FIG. 7 illustrates an assembled view of an autonomous robot (“autobot”) 700 according to the solution comprising an exemplary ultraviolet light subsystem 116 removably mounted to the underside of the autobot body. The exemplary ultraviolet light subsystem 116 shown in the FIG. 7 illustration includes a pair of ultraviolet bulbs 116A, 116B powered by the battery 108, as previously described. The UV light subsystem 116 is configured to generate ultraviolet, type C (UVC) radiation with a predefined power output (or a variable power output, in some embodiments), as previously described, and is operable to remove the pathogens from the target area as the autobot advances along a coverage path.

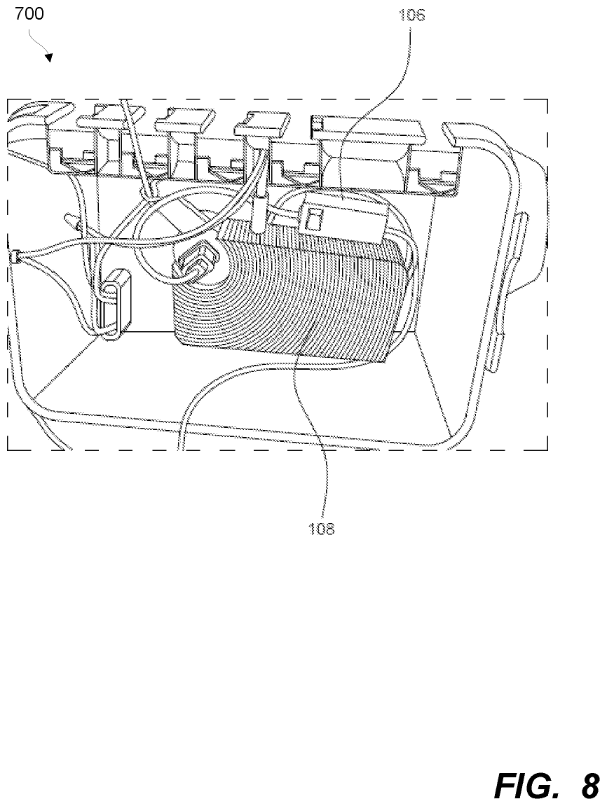

FIG. 8 illustrates an assembled view 800 of an AC/DC converter 106 and a battery 108 comprised within an exemplary embodiment of an autonomous robot (“autobot”) according to the solution.

In the description and claims of the present application, each of the verbs “comprise,” “include” and “have,” and conjugates thereof, are used to indicate that the object or objects of the verb are not necessarily a complete listing of members, components, elements, or parts of the subject or subjects of the verb.

The present solution has been described using detailed descriptions of embodiments thereof that are provided by way of example and are not intended to limit the scope of the solution. The described embodiments comprise different features, not all of which are required in all embodiments of the solution. Some embodiments of the present solution utilize only some of the features or possible combinations of the features. Variations of embodiments of the present solution that are described and embodiments of the present solution comprising different combinations of features noted in the described embodiments will occur to persons of the art.

It will be appreciated by persons skilled in the art that the present invention is not limited by what has been particularly shown and described herein above. Rather the scope of the invention is defined by the claims that follow.

What is claimed is:

1. An autonomous robot (autobot) configured to remove pathogens from a target area, comprising:
a body portion to support a plurality of components of the autobot, wherein the plurality of components comprises:
an AC/DC converter to transform alternating current (AC) input received from an electrical power supply into direct current (DC) output;
a battery to store the direct current (DC) output, wherein the electrical power supply is connected to a charge station to recharge the battery when the autobot is docked at the charge station;
a processor mounted on the body portion to store a plurality of position markers and a plurality of executable instructions pertaining to a coverage path of the target area, wherein the position markers are a plurality of underground reference points positioned along the coverage path;
a sensing system operative to sense the presence of the position markers and transmit data signals associated with the position markers to the processor, wherein the processor operates in response to a received data signal from the sensing system to compare the received data signal with the stored position markers to determine an actual position of the autobot within the target area;
a controller connected to the processor for controlling the movement of the autobot along the coverage path, wherein the processor transmits a correction signal to the controller in response to identifying a deviation of the actual position from the coverage path, wherein the controller adjusts movement of the autobot on receiving the correction signal;
an ultraviolet light subsystem connected to the processor to generate ultraviolet, type C (UVC) radiation with a predefined power output operable to remove the pathogens from the target area as the autobot advances along the coverage path, wherein the processor stores one or more of UVC radiation data and removed pathogens data; and
a transmitter connected to the processor and operable to wirelessly transmit the UVC radiation data and/or removed pathogens data to a remote computing device.

2. The autobot as claimed in claim 1, wherein the controller initiates a return signal to the autobot on detecting a battery level signal that is below a predefined battery level and returns the autobot to the charge station.

3. The autobot as claimed in claim 1 further comprising a dusk-dawn sensor mounted on the body portion to determine an optimal duration to remove the pathogens from a surface of the target area.

4. The autobot as claimed in claim 1 further comprising a Global Positioning System (GPS) connected to the sensing system to establish a geo-fence within which generation of ultraviolet, type C (UVC) radiation occurs.

5. The autobot as claimed in claim 1 further comprising an intensity controller connected to adjust a power output of the ultraviolet light subsystem.

6. The autobot as claimed in claim 1, wherein the battery transmits a battery level signal to the controller.

7. An autonomous robot (autobot) configured to remove pathogens from a target area defined by a geo-fence, comprising:
a body portion to support a plurality of components of the autobot, wherein the plurality of components comprises:
an AC/DC converter to transform alternating current (AC) input received from an electrical power supply into direct current (DC) output;
a battery to store the direct current (DC) output, wherein the electrical power supply is connected to a charge station to recharge the battery when the autobot is docked at the charge station;
a Global Positioning System (GPS) module operable to generate a plurality of GPS coordinates representing a geographical location of the autobot within the target area defined by the geo-fence;
a controller connected to the GPS module and comprising a processor and a memory, wherein the controller is configured to compare the geographical location of the autobot with the GPS coordinates associated with the geo-fence and, based on the comparison, control the movement of the autobot within the target area according to an algorithm for defining a coverage path of the target area; and
an ultraviolet light subsystem configured to generate ultraviolet, type C (UVC) radiation with a predefined power output operable to remove the pathogens from the target area as the autobot advances along the coverage path.

8. The autobot as claimed in claim 7, wherein the processor is further configured to store in the memory one or more of UVC radiation data and removed pathogens data.

9. The autobot as claimed in claim 7 further comprises a transmitter connected to the processor and operable to wirelessly transmit the UVC radiation data and/or removed pathogens data to a remote computing device.

10. The autobot as claimed in claim 7 further comprising a timer module in communication with the controller, wherein the timer module is configured to generate timer signals defining an optimal duration for pathogen removal from the target area and the controller is further configured to, based on the timer signals, cause the autobot to advance along the coverage path or dock to the charge station.

11. The autobot as claimed in claim 10 further comprising a dusk-dawn sensor in communication with the timer module.

12. The autobot as claimed in claim 7, wherein the controller is configured to monitor a battery charge level and, upon determining that the battery charge level is below a predefined threshold, cause the autobot to dock at the charge station.

13. An autonomous robot (autobot) configured to remove pathogens from a target area defined by a geo-fence, comprising:
a body portion to support a plurality of components of the autobot, wherein the plurality of components comprises:
an AC/DC converter to transform alternating current (AC) input received from an electrical power supply into direct current (DC) output;
a battery to store the direct current (DC) output, wherein the electrical power supply is connected to a charge station to recharge the battery when the autobot is docked at the charge station;
a sensing system operative to sense the presence of a boundary wire defining the perimeter of the target area and a charge station guide wire dissecting the target area;
a controller connected to the sensing system and comprising a processor and a memory and configured to control the movement of the autobot within the target area according to an algorithm for defining a coverage path of the target area, wherein the controller is configured to receive signals from the sensing system and, based on the signals, adjust a movement direction of the autobot according to the algorithm; and
an ultraviolet light subsystem configured to generate ultraviolet, type C (UVC) radiation with a predefined power output operable to remove the pathogens from the target area as the autobot advances along the coverage path.

14. The autobot as claimed in claim 13, wherein the processor is further configured to store in the memory component one or more of UVC radiation data and removed pathogens data.

15. The autobot as claimed in claim 13 further comprising a transmitter connected to the processor and operable to wirelessly transmit the UVC radiation data and/or removed pathogens data to a remote computing device.

16. The autobot as claimed in claim 13, further comprising a timer module in communication with the controller, wherein the timer module is configured to generate timer signals defining an optimal duration for pathogen removal from the target area and the controller is further configured to, based on the timer signals, cause the autobot to advance along the coverage path or dock to the charge station.

17. The autobot as claimed in claim 16, further comprising a dusk-dawn sensor in communication with the timer module.

18. The autobot as claimed in claim 13, wherein the controller is configured to monitor a battery charge level and, upon determining that the battery charge level is below a predefined threshold, cause the autobot to dock at the charge station by directing movement of the autobot along the guide wire.
